(19) United States
(12) Patent Application Publication
(10) Pub. No.: US 2019/0202820 A1
(43) Pub. Date: Jul. 4, 2019

(54) PROCESS FOR PRODUCING FLUORINATED COMPOUNDS USING ALCOHOL SOLVENT HAVING CARBONYL GROUP

(71) Applicant: FUTURECHEM CO., LTD., Seoul (KR)

(72) Inventors: Dae Yoon Chi, Seoul (KR); Byoung Se Lee, Gyeonggi-Do (KR); Hyeon Jin Jeong, Seoul (KR); Sung Uk Yang, Seoul (KR)

(21) Appl. No.: 16/042,610

(22) Filed: Jul. 23, 2018

(30) Foreign Application Priority Data
Jan. 2, 2018 (KR) 10-2018-0000442

(51) Int. Cl. C07D 417/04; C07D 221/22; C07H 19/06; C07H 5/02; C07D 233/91

(52) U.S. Cl. CPC C07D 417/04; C07D 221/22; C07D 233/91; C07H 5/02; C07H 19/06

(57) ABSTRACT

The present invention relates to a process for producing an organic fluoro compound containing [¹⁸F]fluorine. The method of the present invention for producing the organic fluoro compound is not only advantageous for producing the organic fluoro compound with high yield by using the solvent represented by formula 1 for nucleophilic fluorination reaction but also suitable for automatic synthesis of ¹⁸F-labeled radiopharmaceuticals due to the excellent solubility of the precursor compound in the solvent.

### CROSS-REFERENCE TO RELATED APPLICATIONS

This application claims priority to Republic of Korea Application No. KR10-2018-0000442, filed Jan. 2, 2018.

### BACKGROUND OF THE INVENTION

### Field of the Invention

The present invention relates to a process for producing an organic fluoro compound containing fluorine-18, which is a radioisotope, using an alcohol solvent having a carbonyl group.

### Description of the Related Art

According to the advancement of modern medical technology, human imaging techniques such as magnetic resonance imaging (MRI), computerized tomography (CT), X-ray imaging, ultrasound imaging, and positron emission tomography (PET) have been developed and used for disease diagnosis.

Unlike other imaging techniques providing anatomical images, positron emission tomography (PET) is able to detect biochemical changes in the body through a radioactive isotope-conjugated molecular probe, which is advantageous in early diagnosis of disease. The radioactive isotope used in positron emission tomography has a short half-life so that radiation exposure is less and it emits gamma ray with high permeability and sensitivity. Therefore, positron emission tomography using such a radioactive isotope is very safe diagnosis method to obtain human body images with a trace amount of toxicity. Various PET radiopharmaceuticals are being developed for diverse diseases, by which images helpful to diagnose such diseases as cancer, cardiovascular disease, and brain disease can be obtained.

Among various positron emitting isotopes, fluorine-18 is the nuclide that is most widely utilized because it has a half-life of 110 minutes suitable for synthesis and use. It can easily be mass-produced using cyclotron, and it is most advantageous because of its low energy and high specific activity.

Fluorine-18 is generally used as [¹⁸F] fluoride in the anion form. [¹⁸F]fluoride ion is a very stable element and is very low in reactivity, making it difficult to bond to organic compounds. [¹⁸F]fluoride can bind to protic hydrogen via strong hydrogen bonding, and thus the nucleophilic properties are reduced. Therefore, the reaction has to be performed under anhydrous conditions.

To increase the reactivity of [¹⁸F]fluoride, an excessive amount of a phase transfer catalyst and a polar aprotic solvent are generally used. However at this time, the reactivity of a base which is added thereto necessarily is also increased, so that various byproducts are generated. To supplement the yield reduction and to solving the problem of the production of byproducts due to the base, a relatively high volume of a precursor is used. However, using a large amount of a precursor makes the product purification difficult.

Fluoride forms a strong hydrogen bond with an alcohol solvent and accordingly the nucleophilicity is significantly reduced. Therefore, such alcohols as methanol and ethanol are not used in the actual reaction. However, such tertiary alcohols such as t-butanol or t-amyl alcohol bind to fluoride via a weak hydrogen bond so that the nucleophilicity can be increased. So, when a tertiary alcohol is used as a reaction solvent, the basicity of the fluoride and the reactivity of the base can be suppressed by a weak hydrogen bond, resulting in the efficient inhibition of side reaction and at the same time resulting in successful link to an organic compound due to the comparatively increased nucleophilicity of the fluoride. [D. W. Kim, D. S. Ahn, Y. H. Oh, S. Lee, H. S. Kil, S. J. Oh, S. J. Lee, J. S. Kim, J. S. Ryu, D. H. Moon, D. Y. Chi. J. Am. Chem. Soc. 2006, 128, 16394]

In the conventional [¹⁸F] fluorination reaction using an aprotic solvent, the yield has been lower because of the side reaction of the base. But, the low reaction yield of [¹⁸F] fluorination reaction using [¹⁸F]FLT and [¹⁸F]FP-CIT can be significantly improved by using a tertiary alcohol solvent. [S. J. Lee, S. J. Oh, D. Y. Chi, S. H. Kang, H. S. Kil, J. S. Kim, D. H. Moon, Nucl. Med. Biol. 2007, 34, 345-351]

¹⁸F-labeled PET radiopharmaceuticals have to be produced through automated synthesis equipments. Reagents and compounds required for the production have to be used in the form of solution.

Even though a tertiary alcohol solvent could make high yield synthesis possible, it has a low solubility so that most of the precursor compounds used in the synthesis of pharmaceuticals cannot be dissolved. This low solubility makes the automatic synthesis of pharmaceuticals difficult, which is a reason of low industrial usability. To solve the low solubility of the tertiary alcohol, another solvent can be additionally added or a heating process can be added before the automatic synthesis for better solubility. However, it not only reduces the production yield but also causes the production failure.

### SUMMARY OF THE INVENTION

It is an object of the present invention to provide a process for producing an organic fluoro compound containing fluorine-18 using an alcohol solvent having a carbonyl group.

To achieve the above object, the present invention provides a process for producing a fluorinated compound, wherein the step of reacting fluoride with a compound having a leaving group (LG) in a solvent is included and the solvent herein is represented by formula 1 below.

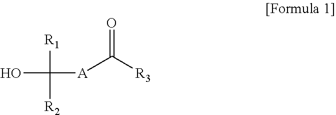

(In formula 1,

R₁ and R₂ are independently hydrogen, or C₁₋₁₀ straight or branched alkyl;

R₃ is C₁₋₁₀ straight or branched alkyl, or C₁₋₁₀ straight or branched alkoxy; and

A is absent, or is C₁₋₄ straight or branched alkylene.)

### Advantageous Effect

The process for producing an organic fluoro compound according to the present invention is advantageous in the preparation of ¹⁸F-labeled radiopharmaceuticals with high yield by using an alcohol solvent having a carbonyl group, compared with the conventional methods using such solvents as t-butanol, t-amyl alcohol and 1-methoxy-2-methyl-2-propanol, wherein the solubility of the precursor compound in the alcohol solvent having the carbonyl group is excellent, indicating that the method is suitable for the automatic synthesis of ¹⁸F-labeled radiopharmaceuticals.

### DESCRIPTION OF THE PREFERRED EMBODIMENTS

Hereinafter, the present invention is described in detail.

The present invention provides a process for producing a fluorinated compound, wherein the step of reacting fluoride with a compound having a leaving group (LG) in a solvent is included and the solvent herein is represented by formula 1 below.

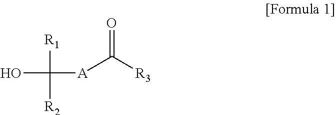

In formula 1,

R₁ and R₂ are independently hydrogen, or C₁₋₁₀ straight or branched alkyl;

R₃ is C₁₋₁₀ straight or branched alkyl, or C₁₋₁₀ straight or branched alkoxy; and

A is absent, or is C₁₋₄ straight or branched alkylene.

Preferably in formula 1 above,

R₁ and R₂ are independently C₁₋₁₀ straight or branched alkyl;

R₃ is C₁₋₁₀ straight or branched alkyl, or C₁₋₁₀ straight or branched alkoxy; and

A is absent, or methylene.

More preferably in formula 1 above,

R₁ and R₂ are independently hydrogen, methyl, or ethyl;

R₃ is methyl, ethyl, methoxy, or ethoxy; and

A is absent, or methylene.

Most preferably in formula 1 above,

R₁ and R₂ are methyl;

R₃ is methyl or methoxy; and

A can be absent.

The fluoride herein can be [¹⁸F]fluoride.

At this time, the [¹⁸F]fluoride herein can be prepared from fluoride salt containing [¹⁸F]fluoride. The fluoride salt above can be selected from the group consisting of fluoride salts of alkali metal such as lithium, sodium, potassium, rubidium, and cesium; fluoride salts of alkali earth metals such as magnesium, calcium, strontium and barium; tetraalkylammonium fluoride; and tetraalkylphosphonium fluoride salt, but not always limited thereto and any known salt form can be used without limitation.

Fluoride anions contained in the fluoride salt can be trapped in a column or cartridge filled with ion exchange solids, and preferably can be captured using QMA (Waters) or Chromafix (Macherey-Nagel). The fluoride anions containing [¹⁸F]fluoride included in the column or cartridge can be eluted by flowing a solution containing a substance selected from the group consisting of tetraalkylammonium salt, tetraalkylphosphonium salt, and cryptofix222-potassium salt dissolved therein on the cartridge. Preferably, a solution containing either tetrabutylammonium salt or cryptofix222-potassium salt dissolved therein is flowed on the cartridge to elute the fluoride anions.

The leaving group (LG) herein can include a halo group or a group represented by formula 2 below.

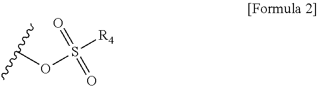

In formula 2 above,

R₄ is -H, nonsubstituted or substituted C₁₋₁₀ straight or branched alkyl, nonsubstituted or substituted C₆₋₁₀ aryl, or nonsubstituted or substituted C₆₋₁₀ aryl C₁₋₃ alkyl,

the substituted alkyl, aryl and aryl alkyl are independently alkyl, aryl and aryl alkyl wherein one or more substituents selected from the group consisting of C₁₋₅ straight or branched alkyl, C₁₋₅ straight or branched alkoxy, halo, amine, nitro, nitrile, and hydroxy are substituted.

Preferably in formula 2 above,

R₄ is —H, nonsubstituted or substituted C₁₋₅ straight or branched alkyl, nonsubstituted or substituted phenyl, or nonsubstituted or substituted phenyl C₁₋₃ alkyl,

the substituted alkyl, phenyl and phenyl alkyl are independently alkyl, phenyl and phenyl alkyl wherein one or more substituents selected from the group consisting of C₁₋₃ straight or branched alkyl, C₁₋₃ straight or branched alkoxy, halo, amine, nitro, nitrile, and hydroxy are substituted.

In addition, the halo can be —F, —Cl, —Br, or —I.

Further, the reaction can be performed for 5 minutes to 60 minutes, for 5 minutes to 55 minutes, for 5 minutes to 50 minutes, for 5 minutes to 45 minutes, for 5 minutes to 40 minutes, for 5 minutes to 35 minutes, for 5 minutes to 30 minutes, for 5 minutes to 25 minutes, for 5 minutes to 20 minutes, for 5 minutes to 15 minutes, or for 5 minutes to 10 minutes, but not always limited thereto. At this time, if the reaction time is less than 5 minutes, there is a problem that the desired ¹⁸F-labeled radiopharmaceutical (fluorinated compound) is not sufficiently produced. On the other hand, if the reaction time is longer than 60 minutes, there is a problem that time is wasted because the reaction is induced more than necessary time to complete the reaction.

In addition, although the reaction can be performed in a temperature range of 90° C. to 160° C., in a temperature range of 90° C. to 155° C., in a temperature range of 90° C. to 150° C., in a temperature range of 90° C. to 145° C., in a temperature range of 90° C. to 140° C., in a temperature range of 95° C. to 160° C., in a temperature range of 100° C. to 160° C., in a temperature range of 105° C. to 160° C., in a temperature range of 110° C. to 160° C., in a temperature range of 115° C. to 160° C., in a temperature range of 120° C. to 160° C., in a temperature range of 100° C. to 155° C., in a temperature range of 110° C. to 150° C., in a temperature range of 115° C. to 145° C., or in a temperature range of 120° C. to 140° C., but not always limited thereto. At this time, if the reaction temperature is less than 90° C., a sufficient reaction cannot be induced. On the other hand, if the reaction temperature is higher than 160° C., by-products can be generated due to a higher temperature than necessary, and the yield of the desired ¹⁸F-labeled radiopharmaceutical product may be lowered.

The process for producing a fluorinated compound of the present invention can be presented by reaction formula I below.

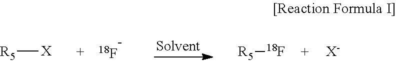

In reaction formula 1,

R₅—X corresponds to a precursor organic compound and can include any known substance that can be used in nucleophilic fluorination reaction. X indicates a leaving group (LG) mentioned above.

R₅ can be an aliphatic compound. At this time, the aliphatic compound includes a ring-free chain compound, a cyclic compound having a ring structure, a saturated compound, and an unsaturated compound. As an example, the aliphatic compound can be a C₁₋₁₀₀ organic compound, a C₁₋₈₀ organic compound, a C₁₋₆₀ organic compound, a C₁₋₄₀ organic compound or a C₁₋₂₀ organic compound.

At this time, one or more carbon atoms constituting the organic compound can be substituted with one or more heteroatoms selected from the group consisting of N, O and S.

Also, one or more hydrogen atoms composing the organic compound can be substituted with one or more substituents, which are exemplified by alkyl, alkoxy, cycloalkyl, aryl, heterocycloalkyl, heteroaryl, cycloalkyl-alkyl, aryl-alkyl, heterocycloalkyl-alkyl, heteroaryl-alkyl, cycloalkyloxy, aryloxy, heterocycloalkyloxy, heteroaryloxy, halogen, hydroxy, nitro, nitrile (cyano), oxo (═O) and carbonyl. At this time, the substituent can be saturated or unsaturated. The unsaturated indicates the status when one or more C═C bonds or one or more C≡C bonds are included or when C═C bonds and C≡C bonds are included together.

Hereinafter, the meaning of each substituent is described in more detail.

alkyl

Alkyl is exemplified by C₁₋₂₃ straight or branched alkyl, C₁₋₁₅ straight or branched alkyl, C₁₋₁₀ straight or branched alkyl, and C₁₋₅ straight or branched alkyl. It also includes unsaturated alkyl containing C═C bond and/or C≡C bond.

One or more carbon atoms constituting alkyl can be substituted with one or more heteroatoms selected from the group consisting of N, O and S.

alkoxy

Alkoxy herein can be represented by “—O-alkyl” and at this time the alkyl is as defined above.

cycloalkyl

Cycloalkyl herein is exemplified by C₃₋₁₅ cycloalkyl, C₃₋₁₀ cycloalkyl, C₃₋₈ cycloalkyl, C₃₋₇ cycloalkyl, C₃₋₆ cycloalkyl, C₃₋₅ cycloalkyl, and C₃₋₄ cycloalkyl. It also includes unsaturated cycloalkyl containing C═C bond and/or C≡C bond.

aryl

Aryl herein is exemplified by C₆₋₁₀ aryl, C₆₋₈ aryl, C₆ aryl, naphthalene, and anthracene.

heterocycloalkyl

When one or more carbon atoms composing cycloalkyl are substituted with one or more heteroatoms selected from the group consisting of N, O and S, it is defined as heterocycloalkyl.

heteroaryl

When one or more carbon atoms composing aryl are substituted with one or more heteroatoms selected from the group consisting of N, O and S, it is defined as heteroaryl

cycloalkyl-alkyl

Cycloalkyl-alkyl can be presented as “-alkyl-cycloalkyl”, and at this time the alkyl and the cycloalkyl are as defined above.

aryl-alkyl

Aryl-alkyl can be presented as “-alkyl-aryl”, and at this time the alkyl and the aryl are as defined above.

heterocycloalkyl-alkyl

Heterocycloalkyl-alkyl can be presented as “-alkyl-heterocycloalkyl”, and at this time the alkyl and the heterocycloalkyl are as defined above.

heteroaryl-alkyl

Heteroaryl-alkyl can be presented as “-alkyl-heteroaryl”, and at this time the alkyl and the heteroaryl are as defined above.

cycloalkyloxy

Cycloalkyloxy can be expressed as “—O-cycloalkyl”, and at this time the cycloalkyl is as defined above.

aryloxy

Aryloxy can be expressed as “—O-aryl”, and at this time the aryl is as defined above.

heterocycloalkyloxy

Heterocycloalkyloxy can be expressed as “—O-heterocycloalkyl”, and at this time the heterocycloalkyl is as defined above.

heteroaryloxy

Heteroaryloxy can be expressed as “—O-heteroaryl”, and at this time the heteroaryl is as defined above.

Halogen can be one or more substances selected from the group consisting of —F, —Cl, —Br and —I; hydroxy indicates —OH; nitro indicates —NO₂; nitrile (cyano) indicates —CN; oxo indicates ═O; and carbonyl indicates C═O.

In addition, the substituents such as alkyl, alkoxy, cycloalkyl, aryl, heterocycloalkyl, heteroaryl, cycloalkyl-alkyl, aryl-alkyl, heterocycloalkyl-alkyl, heteroaryl-alkyl, cycloalkyloxy, aryloxy, heterocycloalkyloxy, heteroaryloxy, halogen, hydroxy, nitro, nitrile (cyano), oxo (═O) and carbonyl explained above can additionally be substituted with other substituents such as alkyl, alkoxy, cycloalkyl, aryl, heterocycloalkyl, heteroaryl, cycloalkyl-alkyl, aryl-alkyl, heterocycloalkyl-alkyl, heteroaryl-alkyl, cycloalkyloxy, aryloxy, heterocycloalkyloxy, heteroaryloxy, halogen, hydroxy, nitro, nitrile (cyano), oxo (═O) and carbonyl explained above, but not always limited thereto.

The fluorinated compound prepared by the process for producing a fluorinated compound of the present invention can include all the random materials informed to those in the art, but preferably can be the compound represented by formula A, B, C, D or E below.

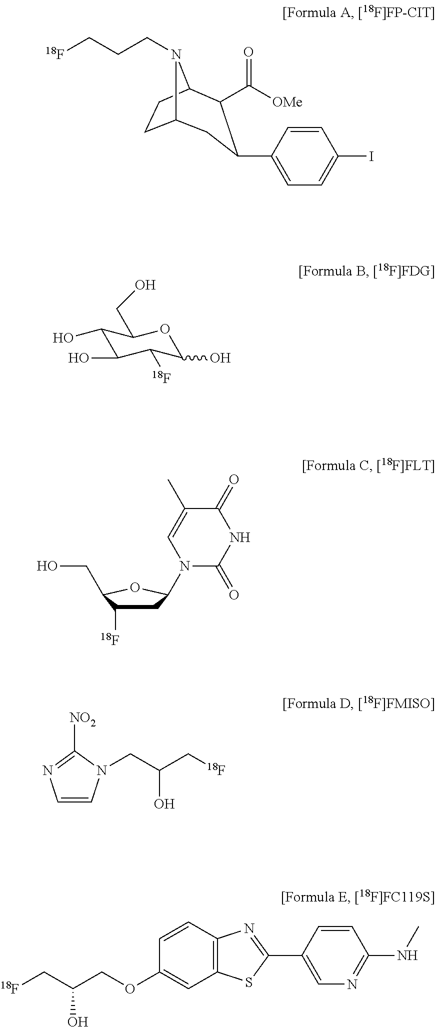

Further, the present invention provides a process for producing ¹⁸F-radiopharmaceuticals with an automatic synthesis apparatus using a precursor organic compound solution prepared by dissolving the compound in an alcohol solvent having a carbonyl group. The process for producing ¹⁸F-radiopharmaceuticals above is substantially the same as the process for producing a fluorinated compound explained above, so that the detailed description thereof will be omitted in order to avoid redundant description.

In the nucleophilic fluorination reaction of the precursor compound, the compound represented by formula 1 is used as a solvent. Since the solvent represented by formula 1 contains an alcohol functional group, the yield of the product is increased by suppressing the side reaction caused by base and the solubility of the precursor compound is increased. The conventional alcohol solvent displays a low solubility of the precursor compound, which is disadvantage because this solvent has to be mixed with another solvent or heated for dissolving the compound. However, the solvent represented by formula 1 of the present invention itself dissolves the precursor compound very well, indicating that it is suitable for the synthesis of ¹⁸F-labeled organic fluoro compounds which requires the use of an automatic synthesis apparatus. Since the compounds and reagents required for the automatic synthesis apparatus have to be used in a solution state, the use of the solvent represented by formula 1 of the present invention enables the automatic synthesis of a stable ¹⁸F-labeled organic fluoro compound. Also, the solvent represented by formula 1 of the present invention is well dissolved in water, so that it is efficiently used for a deprotection reaction using an aqueous solution after the nucleophilic fluorination reaction or a solid phase extraction of the product is facilitated.

To prove the effect of the present invention, the following solvents were used: t-butanol, t-amyl alcohol, 1-methoxy-2-methyl-2-propanol, and an alcohol solvent having a carbonyl group (methyl-2-hydroxyisobutyrate). As a result, it was confirmed that when methyl-2-hydroxyisobutyrate was used, the fluorinated compound can be produced with a remarkably excellent yield (see examples and experimental examples).

Practical and presently preferred embodiments of the present invention are illustrative as shown in the following Examples.

However, it will be appreciated that those skilled in the art, on consideration of this disclosure, may make modifications and improvements within the spirit and scope of the present invention.

### EXAMPLE 1

### Preparation of [¹⁸F]fluoropropylcarbomethoxytropane ([¹⁸F]FP-CIT) using “3-hydroxy-3-methyl-2-butanone” as a solvent

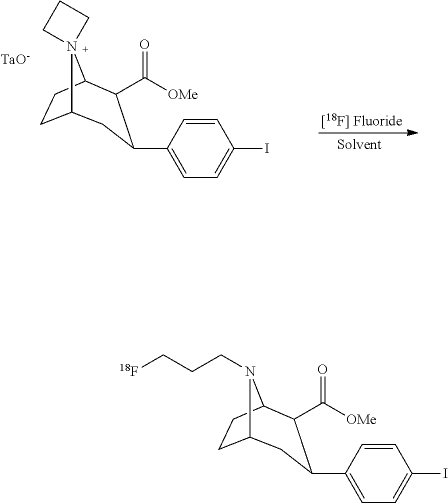

An aqueous solution containing [¹⁸F]fluoride (5˜10 mCi) ions dissolved therein was passed through a cartridge (QMA) filled with an ion exchange resin, and then 3.0 mL of ethanol was spilled thereon. 20 mg of cryptofix222-potassiummethanesulfonate salt (K222-KOMs salt) was dissolved in 1.0 mL of ethanol and the prepared solution was spilled on the cartridge containing [¹⁸F]fluoride trapped therein, followed by elution. The cartridge was heated at 100° C. while nitrogen gas was blown to remove ethanol. 4.0 mg of FP-CIT precursor ((1′R, 2′S, 3′S, 5′S)-3′-(4-iodophenyl)-2′-(methoxycarbonyl)spiro[azetidine-1,8′-bicyclo[3,2,1]octan]-1-ium p-toluenesulfonate), the starting material in the reaction formula above, was loaded in 0.5 mL of 3-hydroxy-3-methyl-2-butanone, followed by reaction at 120° C. for 10 minutes. Radio thin layer chromatography (Radio-TLC) was analyzed at and 10 minutes after the reaction to obtain ¹⁸F-labeled yield.

### EXAMPLE 2

### Preparation of [¹⁸F]fluoropropylcarbomethoxytropane ([¹⁸F]FP-CIT) using “methyl-2-hydroxyisobutyrate” as a solvent

[¹⁸F]fluoropropylcarbomethoxytropane ([¹⁸F]FP-CIT) was prepared by the same manner as described in example 1 except that methyl-2-hydroxyisobutyrate was used as the reaction solvent instead of 3-hydroxy-3-methyl-2-butanone.

### COMPARATIVE EXAMPLE 1

### Preparation of [¹⁸F]fluoropropylcarbomethoxytropane ([¹⁸F]FP-CIT) using “t-butanol” as a solvent

[¹⁸F]fluoropropylcarbomethoxytropane ([¹⁸F]FP-CIT) was prepared by the same manner as described in example 1 except that t-butanol was used as the reaction solvent instead of 3-hydroxy-3-methyl-2-butanone and the reaction was performed at 100° C. instead of 120° C.

### COMPARATIVE EXAMPLE 2

### Preparation of [¹⁸F]fluoropropylcarbomethoxytropane ([¹⁸F]FP-CIT) using “t-amyl alcohol” as a solvent

[¹⁸F]fluoropropylcarbomethoxytropane ([¹⁸F]FP-CIT) was prepared by the same manner as described in example 1 except that t-amyl alcohol was used as the reaction solvent instead of 3-hydroxy-3-methyl-2-butanone and the reaction was performed at 100° C. instead of 120° C.

### COMPARATIVE EXAMPLE 3

### Preparation of [¹⁸F]fluoropropylcarbomethoxytropane ([¹⁸F]FP-CIT) using “1-methoxy-2-methyl-2-propanol” as a solvent

[¹⁸F]fluoropropylcarbomethoxytropane ([¹⁸F]FP-CIT) was prepared by the same manner as described in example 1 except that 1-methoxy-2-methyl-2-propanol was used as the reaction solvent instead of 3-hydroxy-3-methyl-2-butanone.

The solvents used in Examples 1 to 2 and Comparative Examples 1 to 3, the reaction temperature and the production yields after 5 and 10 minutes of the process are shown in Table 1 below.

TABLE 1

| | | Boiling | | Radio-TLC | | |
|---|---|---|---|---|---|---|
| | | point | Temp. | (%) | | |
| | Solvent | (° C.) | (° C.) | 5 min | 10 min | |
| Example 1 | 3-hydroxy-3-methyl- | 140 | 120 | 41 | 48 | Present |
| | 2-butanone | | | | | Invention |
| Example 2 | methyl-2- | 137 | 120 | 81 | 95 | Present |
| | hydroxyisobutyrate | | | | | Invention |
| Comparative | t-butanol | 82 | 100 | 52 | 56 | KR-10- |
| Example 1 | | | | | | 0789847 |
| Comparative | t-amyl alcohol | 102 | 100 | 85 | 84 | KR-10- |
| Example 2 | | | | | | 0789847 |
| Comparative | 1-methoxy-2-methyl- | 118 | 120 | 17 | 17 | KR-10- |
| Example 3 | 2-propanol | | | | | 1605291 |

As shown in Table 1, Comparative Example 1 and Comparative Example 2 present the comparative results corresponding to the prior art (KR 10-0789847), wherein the reaction was induced at 100° C. which was the boiling point of t-butanol and t-amyl alcohol used therein. t-Butanol showed the radio thin layer chromatography yield of 56% after 10 minutes, while t-amyl alcohol showed the yield of 84%. However, t-Butanol and t-amyl alcohol hardly dissolved the precursor compound at room temperature.

Comparative example 3 presents the comparative result corresponding to the prior art (KR 10-1605291), wherein 1-methoxy-2-methyl-2-propanol was used as a solvent and the reaction was induced at 120° C., resulting in 17% of radio thin layer chromatography yield. This result was 5 times lower than the result of the prior art (KR 10-0789847) and approximately 5.6 times lower than the result of Example 2 of the present invention.

Example 1 and Example 2 provide the results of the present invention. Precisely, 3-hydroxy-3-methyl-2-butanone of Example 1 showed the radio thin layer chromatography yield of 56%, while methyl-2-hydroxybutyrate showed the yield of 95%.

The solvents used in Example 1 and Example 2 of the present invention could dissolve the precursor compound well at room temperature.

That is, the solvents used in the prior art such as t-butanol, t-amyl alcohol and 1-methoxy-2-methyl-2-propanol could not dissolve the precursor compound at room temperature (20˜25° C.), while the solvents used in this invention, 3-hydroxy-3-methyl-2-butanone and methyl-2-hydroxyisobutyrate, could dissolve the precursor compound well at room temperature.

The commercial ¹⁸F-labeled radiopharmaceutical has to be synthesized by an automatic synthesis apparatus, and at this time the compounds and the reagents loaded in the automatic synthesis apparatus have to be in a solution state. If the solvent has a low solubility for the target precursor, putting the precursor in a solution state in the automatic synthesis apparatus itself becomes difficult.

The solvent used in the prior art did not dissolve the precursor compound well enough. Therefore, a mixture of the precursor compound and a solvent has to be heated to prepare a solution wherein the precursor compound is dissolved temporarily therein to be loaded in the automatic synthesis apparatus. However, once the solution is cooled down to room temperature after being placed in the automatic synthesis apparatus, the dissolved precursor compound solidifies, and as a result it is clogged in the tube of the apparatus where the solution has to pass and thus the desired amount of the precursor cannot flow in. As a result, the production yield of the ¹⁸F-labeled radiopharmaceuticals is remarkably reduced, resulting in the poor production.

However, when the solvent of the present invention is used to dissolve the precursor compound, it dissolves the precursor compound well at room temperature, so that it can bring a stable production of the ¹⁸F-labeled radiopharmaceuticals.

### EXAMPLE 3

### Preparation of [¹⁸F]fluoropropylcarbomethoxytropane ([¹⁸F]FP-CIT) using “methyl-2-hydroxyisobutyrate” as a solvent (repeat 4 times)

The experiment was repeated four times in the same manner as described in Example 2, and the results are shown in Table 2 below.

TABLE 2

Radio-TLC (%)

| | 5 min | 10 min |
|---|---|---|
| Example 2 | 81 | 95 |
| Repeat 1 | 90 | 95 |
| Repeat 2 | 76 | 89 |
| Repeat 3 | 78 | 88 |
| Repeat 4 | 92 | 95 |
| Mean ± SD | 83.4 ± 7.2 | 92.4 ± 3.6 |

As shown in table 2, from the results of 5 times repeated experiments using methyl-2-hydroxyisobutyrate as a reaction solvent, it was confirmed that the solvent was usable for the synthesis of [¹⁸F]FP-CIT with as significantly high yield as 92% 10 minutes after the reaction.

### EXAMPLE 4

### Preparation of [¹⁸F]FDG (Fluorodeoxyglucose) using “methyl-2-hydroxyisobutyrate” as a solvent

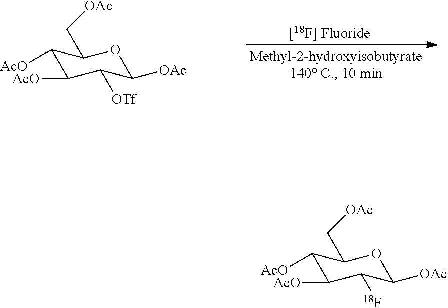 [SMILES: CC(=O)OCC1OC(OC(C)=O)C(OS(=O)(=O)C(F)(F)F)C(OC(C)=O)C1OC(C)=O ; CC(=O)OCC1OC(OC(C)=O)C([18F])C(OC(C)=O)C1OC(C)=O]

An aqueous solution containing [¹⁸F]fluoride (5˜10 mCi) ions dissolved therein was passed through a cartridge (QMA) filled with ion exchange resin to capture the ions, and then passed through 3.0 mL of ethanol. 20 mg of cryptofix222-potassium methanesulfonate salt (K222-KOMs salt) was dissolved in 1.0 mL of ethanol, and this solution was poured into the cartridge containing [¹⁸F]fluoride, followed by elution. Ethanol was removed by blowing nitrogen gas while heating to 100° C. 20 mg of FDG precursor (1,3,4,6-tetra-O-acetyl-2-O-trifluoro-methanesulfonyl-beta-D-mannopyranose) which was the starting material in reaction formula above was dissolved in 1.0 mL of methyl-2-hydroxyisobutyrate, which was added thereto, followed by reaction at 120° C. for 10 minutes. 5 and 10 minutes after the reaction, radio-TLC was analyzed and 69% and 76% yields were confirmed respectively.

### EXAMPLE 5

### Preparation of [¹⁸F]FLT (Fluorothymidine) using “methyl-2-hydroxyisobutyrate” as a solvent

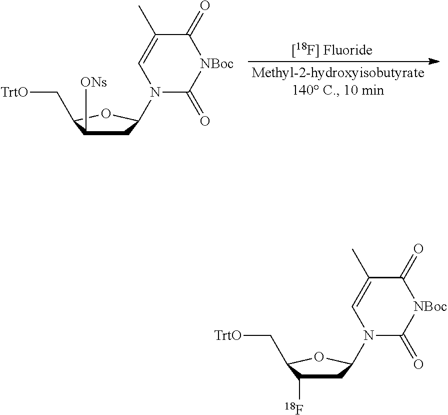

An aqueous solution containing [¹⁸F]fluoride (5˜10 mCi) ions dissolved therein was passed through a cartridge (QMA) filled with ion exchange resin to capture the ions, and then passed through 3.0 mL of ethanol. 20 mg of cryptofix222-potassium methanesulfonate salt (K222-KOMs salt) was dissolved in 1.0 mL of ethanol, and this solution was poured into the cartridge containing [¹⁸F]fluoride, followed by elution. Ethanol was removed by blowing nitrogen gas while heating to 100° C. 20 mg of FLT precursor (3-N-Boc-5′-O-trityl-3′-O-nosyl-thymidine) which was the starting material in reaction formula above was dissolved in 1.0 mL of methyl-2-hydroxyisobutyrate, which was added thereto, followed by reaction at 140° C. for 10 minutes. 5 and 10 minutes after the reaction, radio-TLC was analyzed and 36% and 45% yields were confirmed respectively.

### EXAMPLE 6

### Preparation of [¹⁸F]FMISO (Fluoromisonidazole) using “methyl-2-hydroxyisobutyrate” as a solvent

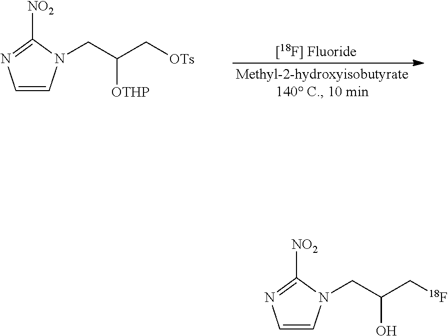

An aqueous solution containing [¹⁸F]fluoride (5˜10 mCi) ions dissolved therein was passed through a cartridge (QMA) filled with ion exchange resin to capture the ions, and then passed through 3.0 mL of ethanol. 20 mg of cryptofix222-potassium methanesulfonate salt (K222-KOMs salt) was dissolved in 1.0 mL of ethanol, and this solution was poured into the cartridge containing [¹⁸F]fluoride, followed by elution. Ethanol was removed by blowing nitrogen gas while heating to 100° C. 20 mg of FMISO precursor (3-(2-nitro-1H-imidazol-1-yl)-2-((tetrahydro-2H-pyran-2-yl)oxy)propyl 4-methylbenzenesulfonate) which was the starting material in reaction formula above was dissolved in 1.0 mL of methyl-2-hydroxyisobutyrate, which was added thereto, followed by reaction at 140° C. for 10 minutes. 5 and 10 minutes after the reaction, radio-TLC was analyzed and 40% and 55% yields were confirmed respectively.

### EXAMPLE 7

### Preparation of [¹⁸F]FC119S using “methyl-2-hydroxyisobutyrate” as a solvent

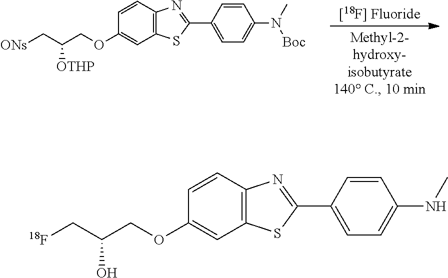

An aqueous solution containing [¹⁸F]fluoride (5˜10 mCi) ions dissolved therein was passed through a cartridge (QMA) filled with ion exchange resin to capture the ions, and then passed through 3.0 mL of ethanol. 20 mg of cryptofix222-potassium methanesulfonate salt (K222-KOMs salt) was dissolved in 1.0 mL of ethanol, and this solution was poured into the cartridge containing [¹⁸F]fluoride, followed by elution. Ethanol was removed by blowing nitrogen gas while heating to 100° C. 5 mg of FC119S precursor ((tert-butoxycarbonyl))(methyl)amino)pyridin-3-yl)benzo[d]thiazol-6-yl)oxy)-2-((tetrahydro-2H-pyran-2-yl)oxypropyl 4-nitrobenzenesulfonate) which was the starting material in reaction formula above was dissolved in 1.0 mL of methyl-2-hydroxyisobutyrate, which was added thereto, followed by reaction at 140° C. for 10 minutes. 5 and 10 minutes after the reaction, radio-TLC was analyzed and 31% and 51% yields were confirmed respectively.

### EXPERIMENTAL EXAMPLE 1

### Comparison of Solubility of the Precursor Compound According to the Kind of Reaction Solvents

The solubility of FP-CIT, FDG, FLT, FMISO, and FC119S at room temperature and at 60° C. according to the different solvents of methyl 2-hydroxyisobutyrate solvent of the present invention and t-amyl alcohol solvent of the prior art (KR 10-0789847) was investigated. Observation with the naked eye was performed and the results are shown in Table 3 below.

TABLE 3

| | | Precursor | Solubility | |
|---|---|---|---|---|
| Reaction | | amount | Room | |
| solvent | Precursor | (mg) | temperature | 60° C. |
| methyl 2- | FP-CIT | 4 | dissolved | dissolved |
| hydroxyisobutyrate | FDG | 20 | dissolved | dissolved |
| (present | FLT | 20 | undissolved | dissolved |
| invention) | FMISO | 5 | dissolved | dissolved |
| | FC119S | 5 | undissolved | dissolved |
| t-amyl alcohol | FP-CIT | 4 | undissolved | dissolved |
| (KR 10-0789847) | FDG | 20 | undissolved | dissolved |
| | FLT | 20 | undissolved | undissolved |
| | FMISO | 5 | undissolved | dissolved |
| | FC119S | 4 | undissolved | undissolved |

As shown in Table 3 above, all the precursors did not dissolve in t-amyl alcohol, the solvent of the prior art, at room temperature. The precursors FLT and FC119S were not dissolved even by heating at 60° C. Those precursors dissolved at 60° C., FP-CIT, FDG and EMISO, were precipitated again as solids when the temperature dropped to room temperature.

On the contrary, when methyl-2-hydroxy isobutyrate, the solvent of the present invention, was used, FP-CIT and FMISO precursors were completely dissolved at room temperature, and FLT and FC119S precursors were also dissolved at 60° C. The FLT and FC119S precursors dissolved in methyl-2-hydroxyisobutyrate remained as a solution without being precipitated as a solid even after cooling to room temperature.

In the preparation of ¹⁸F-radiopharmaceuticals using the automatic synthesis apparatus, the precursor compounds have to be used in a solution state. Most of the precursor compounds were dissolved well in methyl-2-hydroxy isobutyrate, the solvent of the present invention, when it is heated at 60° C. or even at room temperature. When the temperature is dropped to room temperature, the precursor compound solution stayed as a solution state. Therefore, it was confirmed that the present invention using methyl-2-hydroxyisobutyrate as a reaction solvent is efficient in the preparation of ¹⁸F-radiopharmaceuticals using the automatic synthesis apparatus.

### EXPERIMENTAL EXAMPLE 2

### Automatic synthesis of [¹⁸F]FP-CIT (Fluoropropyl-carbomethoxytropane) using methyl-2-hydroxyisobutyrate

The following experiment was performed to prepare [¹⁸F]FP-CIT using an automatic synthesis apparatus under the same conditions as shown in Example 2.

The automatic synthesis apparatus used herein was sCUBE® (CS CHEM). Disposable cassettes and reagent kits according to Example 2 were used. 1.5 mL of methyl-2-hydroxyisobutyrate solution containing 4 mg of FP-CIT precursor dissolved therein was used for the reaction. Upon completion of the reaction, purification was performed by high performance liquid chromatography (HPLC) equipped with sCUBE®, the automatic synthesis apparatus. The isolated [¹⁸F]FP-CIT was diluted in 40 mL of distilled water, which was then absorbed on C-18 cartridge (SePak). The cartridge was washed with distilled water, followed by elution using 2.0 mL of ethanol. This procedure was repeated four more times. The results of the automatic synthesis experiment are summarized in Table 4 below.

TABLE 4

| | Number of times | | | | |
|---|---|---|---|---|---|
| | 1 | 2 | 3 | 4 | 5 |
| Yield | 12.5 | 18.6 | 19.6 | 19.8 | 32.7 |
| (%, attenuation | | | | | |
| correction) | | | | | |

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended Claims.

What is claimed is:

1. A process for producing a fluorinated compound, wherein the step of reacting fluoride with a compound having a leaving group (LG) in a solvent is included and the solvent herein is represented by formula 1 below. 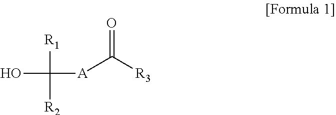
(In formula 1,
R₁ and R₂ are independently hydrogen, or C₁₋₁₀ straight or branched alkyl;
R₃ is C₁₋₁₀ straight or branched alkyl, or C₁₋₁₀ straight or branched alkoxy; and
A is absent, or is C₁₋₄ straight or branched alkylene.)

2. The process for producing a fluorinated compound according to claim 1, wherein R₁ and R₂ are independently C₁₋₁₀ straight or branched alkyl; R₃ is C_{1 10} straight or branched alkyl, or C_{1 10} straight or branched alkoxy; and A is absent, or methylene.

3. The process for producing a fluorinated compound according to claim 1, wherein R₁ and R₂ are independently hydrogen, methyl, or ethyl; R₃ is methyl, ethyl, methoxy, or ethoxy; and A is absent, or methylene.

4. The process for producing a fluorinated compound according to claim 1, wherein R₁ and R₂ are methyl; R₃ is methyl or methoxy; and A can be absent.

5. The process for producing a fluorinated compound according to claim 1, wherein the fluoride is [¹⁸F] fluoride.

6. The process for producing a fluorinated compound according to claim 1, wherein the leaving group includes a halo group or a group represented by formula 2 below. 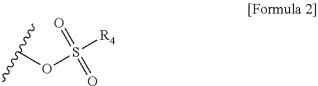
(In formula 2,
R₄ is —H, nonsubstituted or substituted C₁₋₁₀ straight or branched alkyl, nonsubstituted or substituted C₆₋₁₀ aryl, or nonsubstituted or substituted C₆₋₁₀ aryl C₁₋₃ alkyl, and the substituted alkyl, aryl and aryl alkyl are independently alkyl, aryl and aryl alkyl wherein one or more substituents selected from the group consisting of C₁₋₅ straight or branched alkyl, C₁₋₅ straight or branched alkoxy, halo, amine, nitro, nitrile, and hydroxy are substituted.)

7. The process for producing a fluorinated compound according to claim 6, wherein R₄ is —H, nonsubstituted or substituted C₁₋₅ straight or branched alkyl, nonsubstituted or substituted phenyl, or nonsubstituted or substituted phenyl C₁₋₃ alkyl, and the substituted alkyl, phenyl and phenyl alkyl are independently alkyl, phenyl and phenyl alkyl wherein one or more substituents selected from the group consisting of C₁₋₃ straight or branched alkyl, C₁₋₃ straight or branched alkoxy, halo, amine, nitro, nitrile, and hydroxy are substituted.

8. The process for producing a fluorinated compound according to claim 6, wherein the halo group is —F, —Cl, —Br, or —I.

9. The process for producing a fluorinated compound according to claim 1, wherein the reaction is performed for 5˜60 minutes.

10. The process for producing a fluorinated compound according to claim 1, wherein the reaction is performed at 90˜160° C.
